(19) United States
(12) Patent Application Publication
(10) Pub. No.: US 2024/0024519 A1
(43) Pub. Date: Jan. 25, 2024

(54) RADIOPHARMACEUTICAL COMPOSITIONS OF COPPER FOR TARGETED MOLECULAR IMAGING

(71) Applicant: Curium US LLC, St. Louis, MO (US)

(72) Inventors: Amanda Donovan, St. Louis, MO (US); Lauren Radford, St. Louis, MO (US); Allan Casciola, St. Louis, MO (US)

(21) Appl. No.: 18/354,581

(22) Filed: Jul. 18, 2023

Related U.S. Application Data
(60) Provisional application No. 63/390,124, filed on Jul. 18, 2022.

(51) Int. Cl. A61K 51/08; A61K 51/12

(52) U.S. Cl. CPC A61K 51/088; A61K 51/121

(57) ABSTRACT

The present disclosure provides radiopharmaceutical composition comprising copper-64 that performs as a targeted diagnostic PET-agent. The radiopharmaceutical composition disclosed herein has a short physical half-life of about 12.7 h.

### CROSS-REFERENCE TO RELATED APPLICATION

This application claims priority to U.S. Provisional Application No. 63/390,124, filed on Jul. 18, 2022, which is hereby incorporated by reference in its entirety.

### FIELD OF THE INVENTION

The present disclosure relates to a radiopharmaceutical composition comprising copper that performs as a targeted molecular imaging agent for use in positron emission tomography (PET).

### BACKGROUND OF THE INVENTION

Prostate cancer (PC) is the second most common type of cancer among males in the world and the fifth most common cause of cancer-related mortality in males. Determination of serum prostate-specific antigen (PSA) levels is a standard procedure for screening and early detection of PC, but has demonstrated suboptimal diagnostic value. Although an improvement, noninvasive staging using standard imaging modalities, including computed tomography (CT), magnetic resonance imaging (MRI), and bone scintigraphy, provides unsatisfactory results with insufficient sensitivity in detecting remote and lymph node metastases (LNMs), thus leading to a significant underestimation of disease.

Different positron-emitting tracers like C-11 (carbon-11) choline and F-18 (fluorine-18) choline have been studied and found useful for the detection of lesions in PC patients using PET. C-11 or F-18 choline PET/CT imaging has been used to complement PSA levels for early detection of prostate adenocarcinoma; however, numerous clinical studies report low sensitivity and specificity, especially at low PSA levels and high Gleason scores. In addition, C-11 or F-18 choline imaging procedures are flow-limited, and higher uptake may occur in patients with benign prostatic hyperplasia. Although C-11 or F-18 choline PET/CT imaging has good specificity for detecting LNMs, this imaging procedure again shows low sensitivity, ranging from 10% to 73%.

Prostate-specific membrane antigen (PSMA), a membrane-bound type II glycoprotein with an extensive extracellular domain (44-750 amino acids), plays a significant role in prostate carcinogenesis and progression. This transmembrane protein is overexpressed in androgen-dependent and androgen-independent advanced metastatic prostate cancer, schwannoma, tumor neovasculature of many solid tumors, and in certain subtypes of bladder carcinoma, but shows low-level expression in normal prostate cells and organs such as the brain, kidneys, salivary glands, and small intestine. It is this collection of features that makes PSMA an exemplary target for PC diagnosis, therapy, and management.

PSMA-targeted PET/CT imaging has emerged as a highly sensitive method for detection of locally recurrent or metastatic lesions in the context of biochemical recurrence after primary prostate cancer treatment and for localization of primary prostate cancer. A number of gallium-68 (Ga-68)-labeled PSMA ligands have been evaluated clinically, and all appear similarly beneficial in the staging and management of patients with PC.

To date, Ga-68-labeled PSMA ligands have received the greatest attention in helping to manage patients with PC. However, the short half-life of Ga-68 (67.7 minutes) presents logistical limitations. This short half-life limits the application of Ga-68 PSMA to PET centers with nearby preparation capabilities using a germanium-68/gallium-68 generator and analytical groups to provide quality control for the final product.

A PET radionuclide with a longer half-life would provide an opportunity for PET diagnostic centers to utilize the diagnostic and patient management attributes of PSMA imaging. An excellent candidate for this application is copper-64 (Cu-64), which has a longer half-life of 12.7 hours and emits positrons of favorably low energy (Eβ⁺_{avg}=278 keV) compared to the average positron energy emitted by Ga-68 (Eβ⁺_{avg}=830 keV). Lower energy positrons travel shorter distances prior to annihilation, which can improve the resolution of a PET image since the resulting annihilation photons detected by the scanner are generated closer to their point of origin. The longer half-life of Cu-64 improves logistical constraints in both acceptable distances between the Cu-64 radiopharmaceutical manufacturing site and location of patient administration as well as increased flexibility in patient dosing schedules. Thus, the use of Cu-64 as a diagnostic radioisotope may enable delayed imaging time points, providing sufficient time for clearance from background tissues and resulting in increased image contrast and detection of smaller targeted areas.

Thus, there exists a clinical need for an effective diagnostic agent for PC that can overcome the logistical constraints of shorter-lived PET agents, and that may offer improved sensitivity and resolution over similar Ga-68-labeled agents. Cu-64 PSMA I&T injection is a new and promising diagnostic option for patients with PC.

### SUMMARY OF THE INVENTION

Among the various aspects of the present disclosure is a radiopharmaceutical composition comprising copper that performs as an oncologic agent for the diagnosis and staging of PC.

Also disclosed herein are PSMA-specific PET-agents with a moderate half-life, such as copper-64 (⁶⁴Cu or Cu-64, t_{1/2}=12.7 h), that can be distributed nationwide from a centralized manufacturing facility as a readily administered dose. Additional advantages of using these PSMA-specific PET-agents are an improvement of tumor-to-tissue ratios by allowing more time for drug product clearance from non-specific tissues allowing for enhanced image quality and sensitivity, as well as the ability to image a higher number of patients daily in comparison to shorter-lived agents.

Further, disclosed herein are radiopharmaceutical compositions of Cu-64 PSMA I&T, which combine the PET-imaging capabilities of Cu-64 with the PSMA-targeting capability of PSMA I&T to form a diagnostic agent capable of monitoring PSMA expression in vivo.

Other features and aspects of the disclosure are described in detail below.

### BRIEF DESCRIPTION OF THE DRAWINGS

FIG. 1 is a general structure of Cu-64 PSMA I&T. Copper-64 is shown at the position where radiolabeling occurs.

FIG. 2A is a representative radio-HPLC chromatogram of a 10-Ci copper Cu-64 PSMA I&T batch when the reaction is purified.

FIG. 2B is a representative radio-HPLC chromatogram of a 10-Ci copper Cu-64 PSMA I&T batch when the purification step is removed. The primary driver of lower radiochemical purity (“RCP”) in the purified reaction was the increased percentage of the radiolytic impurity at 16.7 min. This impurity was largely absent from the final product when the purification step was removed.

### DETAILED DESCRIPTION OF THE INVENTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents, and patent applications cited herein, whether supra or infra, are hereby incorporated by reference in their entirety.

Headings included herein are simply for ease of reference and are not intended to limit the disclosure in any way.

### I. Definitions

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

When introducing elements of the various embodiment(s) of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately,” when referring to a numerical value, shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors that may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

The term “active agent” or “drug,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The term “half-life,” as used herein in the chemical context, refers to the time it takes for half of the radioactive atoms of a specific radionuclide to decay.

The terms “subject” or “patient” are used interchangeably herein and refer to a vertebrate, preferably a mammal. Mammals include, but are not limited to, humans.

### II. Introduction

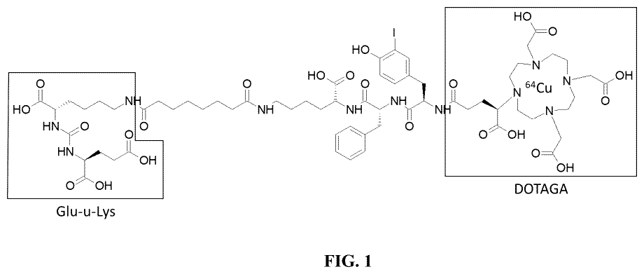

The present disclosure relates to a radiopharmaceutical composition comprising Cu-64 PSMA I&T, which combines the PET-imaging capabilities of Cu-64 with the PSMA-targeting capability of PSMA I&T to form a diagnostic agent capable of monitoring PSMA expression in vivo. Cu-64 PSMA I&T consists of the bioconjugate PSMA I&T radiolabeled with Cu-64, which is bound to PSMA I&T via the bifunctional chelator DOTAGA (FIG. 1).

Cu-64 PSMA I&T injection is provided as a sterile-filtered radiopharmaceutical solution. The chemical name of Cu-64 PSMA I&T injection is: ⁶⁴Cu-(3S, 7S, 26R, 29R, 32R, 37R)-29-benzyl-32(4-hydroxy-3-iodobenzyl)-5, 13, 20, 28, 31, 34-hexaoxo-37-(4,7,10-tris(carboxymethyl)-1, 4, 7, 10-tetraazacyclododecan-1-yl)-4, 6, 12, 21, 27, 30, 33-heptaazaheptatriacontane-1, 3, 7, 26, 37-pentacarboxylic acid. It has the following chemical structure:

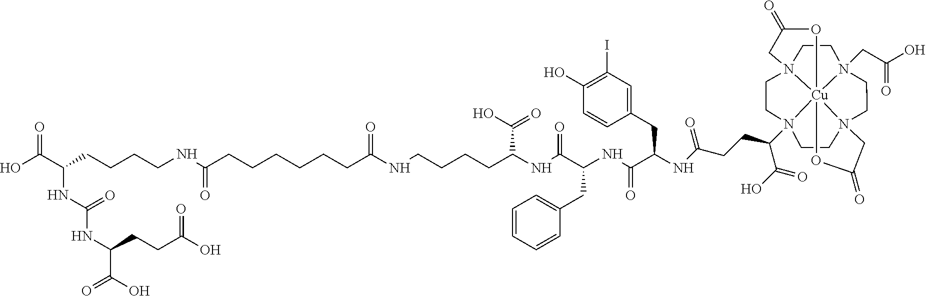

Chemical Structure of Cu-64 PSMA I&T

The invention disclosed herein offers several key advantages. The disclosed Cu-64 PSMA I&T preparation methods and formulations presented herein allow for the preparation of higher activity batch sizes (e.g., up to 60 Ci or 2,220 GBq) and formulation of the final product as a chemically stable and ready-to-administer human dose. In another embodiment, the disclosed Cu-64 PSMA I&T preparation methods and formulations presented herein allow for the preparation of higher activity batch sizes of about 15 Ci to about 60 Ci or 555 GBq to about 2,220 GBq and formulation of the final product as a chemically stable and ready-to-administer human dose. In still another embodiment, the disclosed Cu-64 PSMA I&T preparation methods and formulations presented herein allow for the preparation of higher activity batch sizes of about 15 Ci, about 20 Ci, about 25 Ci, about 30 Ci, about 35 Ci, about 40 Ci, about 45 Ci, about 50 Ci, about 55 Ci, and about 60 Ci and formulation of the final product as a chemically stable and ready-to-administer human dose.

A summary of synthetic and possible formulation production conditions for one embodiment of the present invention is provided for in Table 1.

TABLE 1
Summary of Synthetic and Formulation Production Conditions at Controlled Room Temperature

| Synthetic/Formulation | | |
|---|---|---|
| Production Condition | Parameter | Key Advantage |
| Reaction conditions- | Up to 60,000 mCi | Scaled up reaction size requires the use of |
| maximum radiolabeling | (2,220 GBq) | stabilizing agents in both the reaction |
| activity | | matrix and formulation matrix. Scaling up |
| | | the reaction allows for distribution of |
| | | dozens of patient doses. |
| Reaction conditions- | 21-25° C. for | Decreased reaction temperatures can |
| temperature and time | 5 to 30 min | improve product purity by limiting |
| | | formation of competing metal-PSMA I&T |
| | | complexes. |
| Reaction conditions- | Sodium acetate | Addition of an antioxidant (e.g., gentisic |
| radiolabeling buffer | with gentisic acid | acid) helps prevent radiolytic damage of |
| | | the product and allows production of |
| | | commercial quantities of product with high |
| | | purity. |
| Purification | Optional | Purification can improve radiochemical |
| | | and chemical purity of the finished |
| | | product; however, for higher activity |
| | | batches, purification is more harmful than |
| | | helpful. Disclosed herein are formulations |
| | | wherein high purity is maintained without |
| | | a purification step. |
| Radioactivity | Up to 36 mCi/mL^{a} | Stability at higher starting radioactive |
| concentration (RAC) | | concentration (RAC) extends the shelf life |
| | | of the final product, which is calibrated to |
| | | a specific strength (i.e., radioactive |
| | | concentration) at a fixed time point |
| | | (i.e., calibration time). |
| Product final | Sodium | At higher RAC, saline alone is not |
| formulation | ascorbate/ascorbic | adequate for preventing radiolytic |
| | acid solution | decomposition of the drug. Sodium |
| | | ascorbate behaves as both a pH buffer and |
| | | stabilizer, which allows preparation of |
| | | higher activity amounts and extends |
| | | product shelf life. |

^{a}At time of preparation

Table 2 provides a summary of synthetic and possible formulation production conditions for yet another embodiment of the present invention.

TABLE 2
Summary of Synthetic and Formulation Production Conditions at Either Increased or Decreased Temperatures

| Synthetic/Formulation | | |
|---|---|---|
| Production Condition | Parameter | Key Advantage |
| Reaction conditions- | Up to 60,000 mCi | Scaled up reaction size requires the use of |
| maximum radiolabeling | (2,220 GBq) | stabilizing agents in both the reaction |
| activity | | matrix and formulation matrix. Scaling up |
| | | the reaction allows for distribution of |
| | | dozens of patient doses. |
| Reaction conditions- | 15-90° C. for | Decreased reaction temperatures can |
| temperature and time | 5 to 30 min | improve product purity by limiting |
| | | formation of competing metal-PSMA I&T |
| | | complexes. |
| Reaction conditions- | Sodium acetate | Addition of an antioxidant (e.g., gentisic |
| radiolabeling buffer | with gentisic acid | acid) helps prevent radiolytic damage of |
| | | the product and allows production of |
| | | commercial quantities of product with high |
| | | purity. |
| Purification | Optional | Purification can improve radiochemical |
| | | and chemical purity of the finished |
| | | product; however, for higher activity |
| | | batches, purification is more harmful than |
| | | helpful. Disclosed herein are formulations |
| | | wherein high purity is maintained without |
| | | a purification step. |
| Radioactive | Up to 36 mCi/mL^{a} | Stability at higher starting radioactive |
| concentration (RAC) | | concentration (RAC) extends the shelf life |
| | | of the final product, which is calibrated to |
| | | a specific strength (i.e., radioactive |
| | | concentration) at a fixed time point |
| | | (i.e., calibration time). |
| Product final | Sodium | At higher RAC, saline alone is not |
| formulation | ascorbate/ascorbic | adequate for preventing radiolytic |
| | acid solution | decomposition of the drug. Sodium |
| | | ascorbate behaves as both a pH buffer and |
| | | stabilizer, which allows preparation of |
| | | higher activity amounts and extends |
| | | product shelf life. |

Cu-64 PSMA I&T injection is a PET-agent that specifically targets the prostate-specific membrane antigens that are expressed on metastatic prostate cancer cells. Specifically, Cu-64 PSMA I&T is indicated for the detection and localization of recurrent prostate cancer in males with biochemical recurrence based on elevated blood prostate-specific antigen (PSA) levels following prior treatment. One embodiment of the composition of the drug product is presented in Table 3.

TABLE 3
A representative target composition of a Copper Cu 64 PSMA I&T drug product

| | | | Quantity | | Quantity | |
|---|---|---|---|---|---|---|
| | | Quality | per milliliter | | per unit dose | |
| Name of ingredient(s) | Function | standard | (1 mL) | | (4 mL) | |
| ⁶⁴Cu-PSMA I&T | Drug | GMP | 1.25-2.25 | mCi^{a} | 5-9 | mCi^{a,b} |
| | Substance/Active | | | | | |
| | Pharmaceutical | | | | | |
| | Ingredient | | | | | |
| Ascorbic Acid^{c} | Excipient | USP | 32-39 | mg | 84-148 | mg^{b} |

| | | | | |
|---|---|---|---|---|
| (Sodium Ascorbate) | (pH Buffer and | | | |
| | Stabilizer) | | | |
| Sterile Water for Injection | Excipient (Solvent) | USP | q.s. | q.s. |

q.s: quantum satis (as much as is enough)
^{a}At calibration.
^{b}The patient dose will be either 5 mCi, 7 mCi or 9 mCi;
^{c}Sodium ascorbate is used to formulate solutions for drug product manufacture. The concentration of sodium ascorbate is determined in the drug product and is listed in the specification as ascorbic acid content (target: 37 mg/mL).

Another embodiment of the drug product is presented in Table 4.

TABLE 4
Representative characteristics of a Copper Cu 64
PSMA I&T drug product

| Drug Product Quality | |
|---|---|
| or Attribute | Drug Product Description |
| Appearance | Clear, colorless to yellow solution |
| | without visible particles |
| Radionuclidic identity | Gamma emission spectra at 510-512 |
| | keV and 1344-1348 keV |
| Chemical identity | Relative retention time 1.1 to 1.2 |
| | compared to PSMA I&T in HPLC |
| | standard |
| Radiochemical identity | Relative retention time 1.1 to 1.25 |
| | compared to PSMA I&T in HPLC |
| | standard |
| Radioactive concentration | 2.0-2.5 mCi/mL at calibration |
| Apparent specific activity | ≥90 mCi/mg at calibration |
| Radiochemical purity | ≥95% |
| Chemical impurities | Unspecified impurities: total not more |
| | than the area of PSMA I&T in a |
| | 25 ppm reference solution. |
| | Disregard limit: ≤0.01 times the |
| | A I&T in a 25 ppm reference solution. |
| Gentisic acid content | ≤600 ppm¹ |
| Elemental impurities | USP<232> |
| Residual solvents | USP<467> |
| Dehydrated alcohol (ethanol) | N/A |
| content | |
| Ascorbic acid content | 32-42 mg/mL |
| pH | 5-7 |
| Bacterial endotoxins (BET) | ≤35 EU/mL |
| Sterility | Sterile |
| Filter integrity | Pass |

¹Acceptance criterion is based on max potency limit for a unit dose

In one embodiment, the drug product comprises Copper Cu-64 PSMA I&T in an amount of about 1 mCi, 2 mCi, 3 mCi, 4 mCi, 5 mCi, 6 mCi, 7 mCi, 8 mCi, 9 mCi, or 10 mCi per unit at the time of dose.

### III. Radiopharmaceutical Compositions Comprising Copper

In one embodiment, the medicinal product or radiopharmaceutical composition (or formulation) is a sterile-filtered radiopharmaceutical solution containing a dose of Cu-64 PSMA I&T in an aqueous sodium acetate/gentisic acid solution containing sodium ascorbate/ascorbic acid. The product is diluted to a standard concentration, and therefore, the final volume of the bulk product varies depending on the starting activity introduced.

In another embodiment, the radiopharmaceutical composition or formulation comprises at least one stabilizing agent, a pH adjuster, a metal ion chelator, or a combination thereof.

In another embodiment, one or more stabilizers are selected from the list comprising ethanol, para-aminobenzoic acid (PABA), dihydroxybenzoic acid (gentisate compounds), gentisic acid, cysteine, selenomethionine, ascorbic acid/sodium ascorbate, and methionine.

In another embodiment, pH adjusters are selected from the group consisting of sodium acetate/acetic acid, gentisic acid, ascorbic acid/sodium ascorbate, ammonium acetate, citric acid, sodium citrate, sodium gentisic, and sodium carbonate/bicarbonate.

In one embodiment, the radiopharmaceutical composition or formulation has a purity of at least about 90%, at least about 95%, at least about 97%, or at least about 99% as measured by high-performance liquid chromatography (HPLC), thin-layer chromatography (TLC), instant thin-layer chromatography (iTLC), or gas chromatography (GC). In another embodiment, the radiopharmaceutical composition or formulation has a purity of about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, or about 99.5% as measured by HPLC, TLC, iTLC, or GC.

In another embodiment, the purity of the radiopharmaceutical composition or formulation is measured by HPLC, TLC, or GC at any time post EOS (end of synthesis). In one embodiment, the purity of the radiopharmaceutical composition or formulation is measured by HPLC, TLC, or GC at about 0 hour, about 10 hours, about 15 hours, about 20 hours, about 25 hours, about 30 hours, about 35 hours, about 40 hours, about 45 hours, about 50 hours, about 55 hours, or about 60 hours post EOS. In one particular embodiment, the purity of the radiopharmaceutical composition or formulation is measured by HPLC, TLC, or GC at about 51 hours post EOS.

In one specific embodiment, the radiopharmaceutical composition or formulation has a radiochemical purity of at least about 99% as measured by HPLC, TLC, iTLC, or GC 0 h post EOS. In another specific embodiment, the radiopharmaceutical composition or formulation has a purity of at least about 96.5% as measured by HPLC, TLC, or GC 24 h post EOS, at least about 95% as measured by HPLC, LC, or GC 36 h post EOS, at least about 93% as measured by HPLC, LC, or GC 48 h post EOS, at least about 92.5% as measured by HPLC, LC, or GC 51 h post EOS, at least about 85% as measured by HPLC, LC, or GC 60 h post EOS.

In one embodiment, radionuclidic identity is determined by gamma ray energy detection.

In another embodiment, the radioactivity is measured in a dose calibrator. The chemical amount of Cu-64 PSMA I&T in a dose is calculated from the radioactivity measurement when the dose is dispensed.

In one embodiment, bacterial endotoxin content is determined for each batch before release using a PTS-instrument (USP<85>) and sterility is determined according to USP<71>.

In one embodiment, the radiopharmaceutical composition or formulation is stored at a temperature from about 2° C. to about 55° C., from about 10° C. to about 50° C., from about 15° C. to about 45° C., or from about 20° C. to about 40° C. In one specific embodiment, the radiopharmaceutical composition or formulation is stored at a temperature at about 10° C., about 12.5° C., about 15° C., about 17.5° C., about 20° C., about 20.5° C., about 21° C., about 21.5° C., about 22° C., about 22.5° C., about 23° C., about 23.5° C., about 24° C., about 24.5° C., about 25° C., about 25.5° C., about 26° C., about 26.5° C., about 27° C., about 27.5° C., about 28° C., about 28.5° C., about 29° C., about 29.5° C., about 30° C., about 32.5° C., about 35° C., about 37.5° C., about 40° C., about 42.5° C., about 45° C., about 47.5° C., about 50° C., about 52.5° C., or about 55° C.

In one embodiment, the unit dose is 1 mL, 2 mL, 3 mL, 4 mL, 5 mL, 6 mL, 7 mL, 8 mL, 9 mL, or 10 mL. In a specific embodiment, the unit dose is 4 mL.

i. Cu-64 PSMA I&T

The total amount of labeled Cu-64 PSMA I&T present in the radiopharmaceutical composition can and will vary. In some embodiments, the total amount of Cu-64 PSMA I&T present in the radiopharmaceutical composition may be up to about 50 mCi/mL at EOS. In various embodiments, the total amount of Cu-64 PSMA I&T present in the radiopharmaceutical composition may be about 5 mCi/mL, about 7.5 mCi/mL, about 10 mCi/mL, about 12.5 mCi/mL, about 15 mCi/mL, about 17.5, about 20 mCi/mL, about 22.5 mCi/mL, about 25 mCi/mL, about 27.5 mCi/mL, about 30 mCi/mL, about 32.5 mCi/mL, about 35 mCi/mL, about 37.5 mCi/mL at EOS, about 40 mCi/mL, about 42.5 mCi/mL, about 45 mCi/mL, about 47.5 mCi/mL, or about 50 mCi/mL at EOS. In still other embodiments, the total amount of Cu-64 PSMA I&T present in the radiopharmaceutical composition may be about 30 mCi/mL, about 31 mCi/mL, about 32 mCi/mL, about 33 mCi/mL, about 34 mCi/mL, about 35, about 36 mCi/mL, about 37 mCi/mL, about 38 mCi/mL, about 39 mCi/mL, or about 40 mCi/mL. In another embodiment, the total amount of Cu-64 PSMA I&T present in the radiopharmaceutical composition is about 25 mCi/mL to about 50 mCi/mL at EOS. In yet another embodiment, the total amount of Cu-64 PSMA I&T present in the radiopharmaceutical composition is about 30 mCi/mL to about 45 mCi/mL at EOS. In yet another embodiment, the total amount of Cu-64 PSMA I&T present in the radiopharmaceutical composition is about 35 mCi/mL to about 40 mCi/mL at EOS.

In another embodiment, the total amount of labeled Cu-64 PSMA I&T present in the radiopharmaceutical composition may range from about 1 mCi/mL-3 mCi/mL at calibration. In various embodiments, the total amount of labeled Cu-64 PSMA I&T present at calibration may be about 1 mCi/mL, about 1.25 mCi/mL, about 1.5 mCi/mL, about 1.75 mCi/mL, about 2 mCi/mL, about 2.25 mCi/mL, about 2.5 mCi/mL, 2.75 mCi/mL, or about 3 mCi/mL.

The amount of PSMA I&T present in the radiopharmaceutical composition can also vary. In one embodiment, the amount PSMA I&T present in the radiopharmaceutical composition may range from about 10 μg/mL to about 30 μg/mL PSMA I&T. In various embodiments, the total amount of PSMA I&T may be about 1 μg/mL, about 1.25 μg/mL, about 1.5 μg/mL, about 1.75 μg/mL, about 2 μg/mL, about 2.25 μg/mL, or about 2.5 μg/mL. In another embodiment, the total amount of PSMA I&T may range from 1 μg/mL to about 30 μg/mL, from about 10 μg/mL to about 30 μg/mL, from about 15 μg/mL to about 27.5 μg/mL, or from about 20 μg/mL to about 25 μg/mL. In another embodiment, the total amount of PSMA I&T may range from about 22.5 μg/mL to about 27.5 μg/mL.

In one embodiment, the radioactivity of the Cu-64 PSMA I&T in the radiopharmaceutical composition is less than about 150 mCi, less than about 140 mCi, less than about 130 mCi, less than about 120 mCi, less than about 110 mCi, less than about 100 mCi, less than about 90 mCi, less than about 80 mCi, less than about 70 mCi, less than about 60 mCi, less than about 50 mCi, less than about 40 mCi, less than about 30 mCi, less than about 20 mCi, or less than about 10 mCi per unit dose. In another embodiment, the radioactivity of the Cu-64 PSMA I&T in the radiopharmaceutical composition is from about 1 mCi to about 150 mCi, from about 1 mCi to about 125 mCi, from about 1 mCi to about 100 mCi, from about 1 mCi to about 75 mCi, from about 1 mCi to about 50 mCi, from about 1 mCi to about 40 mCi, from about 1 mCi to about 30 mCi, from about 1 mCi to about 20 mCi, from about 1 mCi to about 10 mCi, or from about 4 mCi to about 10 mCi, or from about 5 mCi to about 9 mCi, per unit dose. In one specific embodiment, the radioactivity of the Cu-64 PSMA I&T in the radiopharmaceutical composition is about 1 mCi, about 2 mCi, about 3 mCi, about 4 mCi, about 5 mCi, about 6 mCi, about 7 mCi, about 8 mCi, about 9 mCi, about 10 mCi, about 11 mCi, about 12 mCi, about 13 mCi, about 14 mCi, about 15 mCi, about 18 mCi, about 21 mCi, about 24 mCi, about 27 mCi, about 30 mCi, about 40 mCi, about 50 mCi, about 60 mCi, about 70 mCi, about 80 mCi, about 90 mCi, about 100 mCi, about 110 mCi, about 120 mCi, about 130 mCi, about 140 mCi, or about 150 mCi per unit dose.

In another embodiment, the radioactivity of the Cu-64 PSMA I&T in the radiopharmaceutical composition is less than about 50 mCi, less than about 40 mCi, less than about 30 mCi, less than about 20 mCi, less than about 10 mCi, less than about 5 mCi, less than about 4 mCi, less than about 3 mCi, less than about 2.5 mCi, less than about 2 mCi, less than about 1.5 mCi, or less than about 1 mCi per 1 mL. In another embodiment, the radioactivity of the Cu-64 PSMA I&T in the radiopharmaceutical composition is from about 1 mCi to about 100 mCi, from about 1 mCi to about 50 mCi, from about 1 mCi to about 20 mCi, or from about 1 mCi to about 10 mCi per 1 mL. In one specific embodiment, the radioactivity of the Cu-64 PSMA I&T in the radiopharmaceutical composition is about 1 mCi, about 1.25 mCi, about 1.5 mCi, about 1.75 mCi, about 2 mCi, about 2.25 mCi, about 2.5 mCi, about 2.75 mCi, about 3 mCi, about 3.25 mCi, about 3.5 mCi, about 3.75 mCi, about 4 mCi, about 4.25 mCi, about 4.5 mCi, about 4.75 mCi, about 5 mCi, about 7 mCi, about 9 mCi, about 12 mCi, about 15 mCi, about 18 mCi, about 21 mCi, about 24 mCi, about 27 mCi, about 30 mCi, about 40 mCi, or about 50 mCi per 1 mL.

In one embodiment, the RAC of the Cu-64 PSMA I&T in the radiopharmaceutical composition is less than about 50 mCi/mL, less than about 45 mCi/mL, less than about 40 mCi/mL, less than about 35 mCi/mL, less than about 30 mCi/mL, less than about 25 mCi/mL, less than about 20 mCi/mL, less than about 15 mCi/mL, less than about 10 mCi/mL, less than about 5 mCi/mL, less than about 4 mCi/mL, less than about 3 mCi/mL, or less than about 2 mCi/mL.

In still another embodiment, the RAC of the Cu-64 PSMA I&T in the radiopharmaceutical composition is up to about 40 mCi/mL, up to about 39 mCi/mL, up to about 38 mCi/mL, up to about 37 mCi/mL, up to about 36 mCi/mL, up to about 35 mCi/mL, up to about 34 mCi/mL, up to about 33 mCi/mL, up to about 32 mCi/mL, up to about 31 mCi/mL, up to about 30 mCi/mL, up to about 29 mCi/mL, up to about 28 mCi/mL, up to about 27 mCi/mL, up to about 26 mCi/mL, up to about 25 mCi/mL, up to about 24 mCi/mL, up to about 23 mCi/mL, up to about 22 mCi/mL, up to about 21 mCi/mL, up to about 20 mCi/mL, up to about 19 mCi/mL, up to about 18 mCi/mL, up to about 17 mCi/mL, up to about 16 mCi/mL, up to about 15 mCi/mL, 14 mCi/mL, up to about 13 mCi/mL, up to about 12 mCi/mL, up to about 11 mCi/mL, up to about 10 mCi/mL, up to about 9 mCi/mL, up to about 8 mCi/mL, up to about 7 mCi/mL, up to about 6 mCi/mL, up to about 5 mCi/mL, up to about 4 mCi/mL, up to about 3 mCi/mL, up to about 2 mCi/mL, or up to about 1 mCi/mL.

In another embodiment, the RAC of the Cu-64 PSMA I&T in the radiopharmaceutical composition is from about 1 mCi/mL to about 100 mCi/mL, about 2 mCi/mL to about 90 mCi/mL, about 3 mCi/mL to about 80 mCi/mL, about 4 mCi/mL to about 60 mCi/mL, about 5 mCi/mL to about 50 mCi/mL, from about 10 mCi/mL to about 40 mCi/mL, about 1 mCi/mL to about 40 mCi/mL, about 1 mCi/mL to about 36 mCi/mL, about 1 mCi/mL to about 35 mCi/mL, or about 20 mCi/mL to about 35 mCi/mL. In one specific embodiment, the RAC of the Cu-64 PSMA I&T in the radiopharmaceutical composition is about 1 mCi/mL, about 10 mCi/mL, about 13.5 mCi/mL, about 15 mCi/mL, about 20 mCi/mL, about 27 mCi/mL, about 30 mCi/mL, about 33 mCi/mL, about 35 mCi/mL, about 40 mCi/mL, about 45 mCi/mL, or about 50 mCi/mL.

In yet another embodiment, the Cu-64 PSMA I&T drug product has a standard concentration at the end of production of about 15 mCi/mL to about 40 mCi/mL at the end of production.

In one embodiment, the mass of radioactive pharmaceutical ingredient (Cu-64 PSMA I&T) in the drug product and related non-radioactive substances is less than about 200 μg, less than about 175 μg, less than about 150 μg, less than about 125 μg, less than about 120 μg, less than about 115 μg, less than about 110 μg, less than about 105 μg, less than about 100 μg, less than about 75 μg, or less than about 50 μg per vial.

ii. Antioxidants

In one embodiment, the formulation comprises an antioxidant. In another embodiment, the antioxidant is ascorbate/ascorbic acid. In another embodiment, the antioxidant is gentisic acid. In one specific embodiment, the antioxidant is sodium acetate/gentisic acid solution.

In another embodiment, the total amount of sodium acetate/gentisic acid solution in the radiopharmaceutical composition can and will vary. In some embodiments, sodium acetate/gentisic acid solution present in the radiopharmaceutical composition may range from about 0 μg/mL to about 600 μg/mL or from about 130 μg/mL to about 320 μg/mL of gentisic acid, and about 0 mg/mL to about 3.5 mg/mL or from about 1.3 mg/mL to about 3.3 mg/mL of sodium acetate.

In another embodiment, the concentration of sodium acetate in the final radiopharmaceutical composition may be from about 0.2 M to about 0.5 M, from about 0.25 M to about 0.4 M, or from about 0.3 M to about 0.35 M.

In yet another embodiment, the concentration of sodium acetate in the final radiopharmaceutical composition may be from about 1 mg/mL to about 5 mg/mL, from about 2 mg/mL to about 4 mg/mL, or from about 2.5 mg/mL to about 3.5 mg/mL.

In the final radiopharmaceutical composition dose, the concentration of gentisic acid may range from about 0 mg/mL to about 3.5 mg/mL. In another embodiment, the concentration of gentisic acid may range from about 1.3 mg/mL to about 3.3 μg/mL.

In the final radiopharmaceutical composition dose, the concentration of gentisic acid may range from about 0 mg/mL to about 600 μg/mL. In another embodiment, the concentration of gentisic acid may range from about 130 mg/mL to about 320 μg/mL.

iii. Stabilizing Agent

In one embodiment, the formulation comprises stabilizing agent.

In another embodiment, the stabilizing agent is sodium ascorbate. The total amount of sodium ascorbate in the radiopharmaceutical composition can and will vary. In some embodiments, sodium ascorbate present in the radiopharmaceutical composition may range from about 5 mg to about 300 mg per unit dose. In one embodiment, sodium ascorbate is present in the radiopharmaceutical composition in an amount of about 10 mg, about 20 mg, about 30 mg, about 40 mg, about 50 mg, about 60 mg, about 70 mg, or about 75 mg per 1 mL. In still another embodiment, sodium ascorbate is present in the radiopharmaceutical composition in an amount of about 30 mg, about 31 mg, about 32 mg, about 33 mg, about 34 mg, about 35 mg, about 36 mg, about 37 mg, about 38 mg, about 39 mg or about 40 mg per 1 mL. In yet another embodiment, sodium ascorbate is present in the radiopharmaceutical composition in an amount from about 1 mg to about 500 mg, from about 5 mg to about 300 mg, from about 20 mg to about 80 mg, from about 30 mg to about 60 mg, from about 35 mg to about 50 mg, or from about 40 mg to about 45 mg per 1 mL.

One aspect of the disclosure provides for a radiopharmaceutical composition with a pH from about 3 to about 9, from about 4 to about 9, from about 5 to about 9, from about 3 to about 8, from about 4 to about 8, from about 3 to about 7.5, from about 5 to about 7.5, or from about 5.5 to about 7.5. The pH of the radiopharmaceutical composition may be about 3.0, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

### IV. Preparation of Cu-64 PSMA I&T Drug Product

In one specific embodiment, Cu-64 PSMA I&T is prepared using the general process described herein, which can be performed manually or via automated synthesis. Lyophilized PSMA I&T is reconstituted in a pH-buffered solution of gentisic acid in sodium acetate. The reconstituted PSMA I&T solution is mixed with a solution of ⁶⁴Cu copper chloride and the mixture is heated to 30° C. or held at ambient temperature for up to 30 minutes. The resulting Cu-64 PSMA I&T product can be purified via Cis solid-phase extraction (SPE) or the reaction mixture can be used directly in the next process step (i.e., final formulation). For SPE purification, the drug substance is eluted in a 50% aqueous ethanol solution and the eluate is passed through a 0.2 μm sterilizing filter into a vial containing a solution of aqueous sodium ascorbate. The radioactive concentration of the Cu-64 PSMA I&T solution is adjusted by diluting with additional sodium ascorbate if necessary, after which the bulk drug product solution can be aseptically dispensed.

i. Ligand

The present invention includes ligands that are capable of being chelated to Cu-64. In one embodiment, the ligand is PSMA I&T.

The amount of ligand that is added to a reaction mixture containing Cu-64 varies depending on the amount of activity of Cu-64 present. In one embodiment where the reaction mixture contains about 15 Ci Cu-64, the ligand is added to the reaction mixture in an amount from about 8,000 μg to about 10,000 μg. In another embodiment, about 9,000 μg is added to the reaction mixture.

In another embodiment where the reaction mixture contains about 15 Ci Cu-64, the ligand is added to the reaction mixture in an amount from about 7,000 μg to about 11,000 μg, from about 8,000 μg to about 10,000 μg, or about 9,000 μg is added to the reaction mixture.

In another embodiment where the reaction mixture contains about 20 Ci Cu-64, the ligand is added to the reaction mixture in an amount from about 8,000 μg to about 12,000 μg, from about 9,000 μg to about 11,000 μg, or about 10,000 μg is added to the reaction mixture.

In another embodiment where the reaction mixture contains about 50 Ci Cu-64, the ligand is added to the reaction mixture in an amount from about 20,000 μg to about 30,000 μg, from about 22,000 μg to about 28,000 μg, from about 24,000 μg to about 26,000 μg, or about 25,000 μg is added to the reaction mixture.

In still another embodiment where the reaction mixture contains about 90 Ci Cu-64, the ligand is added to the reaction mixture in an amount from about 36,000 μg to about 54,000 μg, from about 40,000 μg to about 50,000 μg, or about 45,000 μg is added to the reaction mixture.

In another embodiment, PSMA I&T is added to the reaction mixture in an amount from about 1,000 μg to about 60,000 μg, from about 5,000 μg to about 55,000 μg, from about 7,000 μg to about 11,000 μg, from about 8,000 μg to about 12,000 μg, from about 8,000 μg to about 10,000 μg, from about 9,000 μg to about 11,000 μg, from about 20,000 μg to about 30,000 μg, from about 22,000 μg to about 28,000 μg, from about 24,000 μg to about 26,000 μg, from about 36,000 μg to about 54,000 μg, or from about 40,000 μg to about 50,000 μg. In another embodiment, PSMA I&T is added to the reaction mixture in an amount of about 9,000 μg, about 10,000 μg, about 25,000 μg, or about 45,000 μg. In yet another embodiment, the PSMA I&T is added to the reaction mixture in an amount of less than about 11,000 μg, less than about 30,000 μg, or less than about 55,000 μg.

In a further embodiment, PSMA I&T is added to the reaction mixture in an amount of about 9,000 μg for about a 15 Ci batch. In another embodiment, PSMA I&T is added to the reaction mixture in an amount of about 8,000 μg, of about 9,000 μg, of about 10,000 μg, or of about 11,000 μg for about a 20 Ci batch. In still another embodiment, PSMA I&T is added to the reaction mixture in an amount of about 8,000 μg to about 11,000 μg for about a 20 Ci batch. In yet another embodiment, PSMA I&T is added to the reaction mixture in an amount of about 20,000 μg to about 30,000 μg for about a 50 Ci batch. In still another embodiment, PSMA I&T is added to the reaction mixture in an amount of about 36,000 μg to about 54,000 μg for about a 90 Ci batch.

In another embodiment, PSMA I&T is used in an amount from about 0.1 μg/mCi to about 20 μg/mCi, from about 0.5 μg/mCi to about 15 μg/mCi, from about 1 μg/mCi to about 11 μg/mCi, from about 1 μg/mCi to about 8 μg/mCi, from about 1 μg/mCi to about 5 μg/mCi, from about 1 μg/mCi to about 3 μg/mCi, or from about 0.1 μg/mCi to about 1.5 μg/mCi. In yet another embodiment, the PSMA I&T is used in an amount of about 0.1 μg/mCi, about 0.25 μg/mCi, about 0.4 μg/mCi, about 0.5 μg/mCi, about 0.6 μg/mCi, about 0.75 μg/mCi, about 0.8 μg/mCi, about 1 μg/mCi, about 1.25 μg/mCi, about 1.5 μg/mCi, about 1.75 μg/mCi, about 2 μg/mCi, about 2.5 μg/mCi, about 3 μg/mCi, about 3.5, or about 4 μg/mCi.

In one embodiment, the concentration of PSMA I&T per mL in the radiolabeling step is greater than about 100 μg/mL, greater than about 150 μg/mL, greater than about 200 μg/mL, greater than about 250 μg/mL, greater than about 300 μg/mL, greater than about 333 μg/mL, or greater than about 400 μg/mL. In still another embodiment, the concentration of PSMA I&T per mL in the radiolabeling step is greater than about 100 μg/mL, greater than about 110 μg/mL, greater than about 120 μg/mL, greater than about 130 μg/mL, greater than about 140 μg/mL, greater than about 150 μg/mL, greater than about 160 μg/mL, greater than about 170 μg/mL, greater than about 180 μg/mL, greater than about 190 μg/mL, or greater than about 200 μg/mL.

ii. Radionuclide

In one embodiment, ⁶⁴CuCl₂ is added to the reaction mixture (as a source of ⁶⁴Cu) in an amount from about 100 mCi to about 5000 mCi, from about 200 mCi to about 4000 mCi, from about 300 mCi to about 3500 mCi, from about 400 mCi to about 3000 mCi, from about 500 mCi to about 2500 mCi, from about 500 mCi to about 15,000 mCi, from about 1,000 mCi to about 10,000 mCi, from about 1,000 mCi to about 15,000 mCi, or from about 7,500 mCi to about 15,000 mCi. In another embodiment, ⁶⁴CuCl₂ is added to the reaction mixture (as a source of ⁶⁴Cu) in an amount up to about 10,000 mCi. In yet another embodiment, ⁶⁴CuCl₂ is added to the reaction mixture (as a source of ⁶⁴Cu) in an amount up to about 15,000 mCi. In a further embodiment, ⁶⁴CuCl₂ is added to the reaction mixture (as a source of ⁶⁴Cu) in an amount of about 100 mCi, about 200 mCi, about 300 mCi, about 400 mCi, about 500 mCi, about 600 mCi, about 700 mCi, about 800 mCi, about 900 mCi, about 1000 mCi, about 1500 mCi, about 2000 mCi, about 2500 mCi, about 3000 mCi, about 3500 mCi, about 4000 mCi, about 4500 mCi, about 5000 mCi, about 5500 mCi, about 6000 mCi, about 6500 mCi, about 7000 mCi, about 7500 mCi, about 8000 mCi, about 8500 mCi, about 9000 mCi, about 9500 mCi, about 10,000 mCi, about 15,000 mCi, about 20,000 mCi, about 30,000 mCi, about 40,000 mCi, about 50,000 mCi, about 60,000 mCi, about 70,000 mCi, about 80,000 mCi, about 90,000 mCi, or about 100,000 mCi. In yet another embodiment, ⁶⁴CuCl₂ is added to the reaction mixture (as a source of ⁶⁴Cu) in an amount of less than about 100 mCi, less than about 200 mCi, less than about 300 mCi, less than about 400 mCi, less than about 500 mCi, less than about 600 mCi, less than about 700 mCi, less than about 800 mCi, less than about 900 mCi, less than about 1000 mCi, less than about 1500 mCi, less than about 2000 mCi, less than about 2500 mCi, less than about 3000 mCi, less than about 3500 mCi, less than about 4000 mCi, less than about 4500 mCi, less than about 5000 mCi, less than about 5500 mCi, less than about 6000 mCi, less than about 6500 mCi, less than about 7000 mCi, less than about 7500 mCi, less than about 8000 mCi, less than about 8500 mCi, less than about 9000 mCi, less than about 9500 mCi, or less than about 10,000 mCi.

In another embodiment, non-carrier copper, such as CuCl₂, is added to the reaction mixture to improve the consistency of the radiochemical purity of the precursor formulation. This process is referred to herein as “spiking.” In one embodiment, there is at least about 5 ppm of non-carrier copper in the precursor formulation. In another embodiment, there is at least about 1 ppm, about 2 ppm, about 3 ppm, about 4 ppm, about 5 ppm, about 10 ppm, about 15 ppm, about 20 ppm, about 25 ppm, or about 30 ppm of non-carrier copper in the precursor formulation. In still a further embodiment, the amount of non-carrier copper in the precursor formulation is about 5 ppm to about 30 ppm. In still a further embodiment, the amount of non-carrier copper in the precursor formulation is less than about 30 ppm.

iii. Buffer Solution

In one embodiment, the buffer solution used in the preparation of the bulk solution of the drug product is sodium acetate buffer, sodium acetate/gentisic acid buffer, sodium ascorbate buffer, sodium ascorbate/ethanol buffer, ammonium acetate buffer, ammonium acetate/gentisic acid buffer, ammonium ascorbate buffer, ammonium ascorbate/ethanol buffer, sodium phosphate/sodium diphosphate, sodium citrate/citric acid or any other appropriate buffer. In one embodiment, the concentration of gentisic acid in the buffer is from about 1 mg/mL to about 50 mg/mL, from about 1 mg/mL to about 30 mg/mL, from about 1 mg/mL to about 10 mg/mL, or from about 1 mg/mL to about 50 mg/mL. In yet another embodiment, the concentration of gentisic acid in the buffer is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 20 mg/mL, about 30 mg/mL, about 40 mg/mL, or about 50 mg/mL.

In another embodiment, the concentration of sodium acetate in the buffer is from about 10 mg/mL to about 100 mg/mL, from about 20 mg/mL to about 80 mg/mL, from about 30 mg/mL to about 60 mg/mL, or from about 40 mg/mL to about 60 mg/mL. In yet another embodiment, the concentration of sodium acetate in the buffer is about 10 mg/mL, about 20 mg/mL, about 30 mg/mL, about 40 mg/mL, about 45 mg/mL, about 50 mg/mL, about 54.8 mg/mL, about 55 mg/mL, about 60 mg/mL, about 70 mg/mL, about 80 mg/mL, about 90 mg/mL, or about 100 mg/mL.

In another embodiment, the buffer comprises about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL or about 100 mg/mL gentisic acid and about 10 mg/mL, about 20 mg/mL, about 30 mg/mL, about 40 mg/mL, about 45 mg/mL, about 50 mg/mL, about 54.8 mg/mL, about 55 mg/mL, about 60 mg/mL, about 70 mg/mL, about 80 mg/mL, about 90 mg/mL, or about 100 mg/mL of sodium acetate.

In one embodiment, the buffer is a solution containing between about 2 mg/mL to about 4 mg/mL gentisic acid and between about 0.25 M to about 0.4 M sodium acetate. In another embodiment, the buffer is a solution containing about 4 mg/mL of gentisic acid and about 55 mg/mL of sodium acetate. In another embodiment, the buffer solution has a pH of about 4.5 to about 5.5.

iv. Stabilizers

In one embodiment, the stabilizer is gentisic acid. In another embodiment, the stabilizer is ascorbate. However, any appropriate stabilizer may be used.

In another embodiment, more than one stabilizer is used. In another embodiment, one stabilizer, such as gentisic acid, is used during the radiolabeling process, and another stabilizer, such as sodium ascorbate or ascorbic acid, is used in the final formulated product.

In yet another embodiment, the stabilizer or a combination thereof is added to the reaction mixture in a concentration of about 4 mg/mL, about 10 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL.

In one embodiment, sodium ascorbate is added to the reaction mixture in a concentration of about 1 mg/mL, about 2 mg/mL, about 3 mg/mL, about 4 mg/mL, about 5 mg/mL, about 10 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL.

In another embodiment, ascorbic acid is added to the reaction mixture in a concentration of about 1 mg/mL, about 2 mg/mL, about 3 mg/mL, about 4 mg/mL, about 5 mg/mL, about 10 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL.

v. Radiolabeling Conditions

In yet another embodiment, the radiolabeling reaction conditions vary. In one embodiment, the radiolabeling reaction is carried at a temperature from about 20° C. to about 95° C., about 20° C. to about 90° C., about 20° C. to about 40° C., about 20° C. to about 35° C., about 35° C. to about 50° C., about 50° C. to about 70° C., about 70° C. to about 95° C., or about 80° C. to about 90° C. In one specific embodiment, the radiolabeling reaction is carried at a temperature of about 10° C., about 12.5° C., about 15° C., about 17.5° C., about 20° C., about 20.5° C., about 21° C., about 21.5° C., about 22° C., about 22.5° C., about 23° C., about 23.5° C., about 24° C., about 24.5° C., about 25° C., about 25.5° C., about 26° C., about 26.5° C., about 27° C., about 27.5° C., about 28° C., about 28.5° C., about 29° C., about 29.5° C., about 30° C., about 32.5° C., about 35° C., about 37.5° C., about 40° C., about 42.5° C., about 45° C., about 47.5° C., about 50° C., about 52.5° C., about 55° C., about 57.5° C., about 60° C., about 62.5° C., about 65° C., about 67.5° C., about 70° C., about 72.5° C., about 75° C., about 77.5° C., about 80° C., about 82.5° C., about 85° C., about 87.5° C., or about 90° C.

In one embodiment, the radiolabeling reaction time is less than about 60 minutes, less than about 45 minutes, less than about 30 minutes, less than about 15 minutes, or less than about 5 minutes. In another embodiment, the radiolabeling reaction time is about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In yet another embodiment, the radiolabeling reaction time is from about 1 minute to about 60 minutes, from about 5 minutes to about 50 minutes, from about 5 minutes to about 30 minutes, from about 10 minutes to about 40 minutes, or from about 20 minutes to about 30 minutes.

In another embodiment, the pH of the final product is from about 4.0 to 8.5, from about 4.5 to about 8.0, from about 5.0 to about 7.5, or from about 5.5 to about 7.5.

In one embodiment, at the end of production, the activity range for a unit dose containing the radiopharmaceutical composition is about 60 mCi/dose, 70 mCi/dose, 80 mCi/dose, 90 mCi/dose, 100 mCi/dose, 110 mCi/dose, 120 mCi/dose, 130 mCi/dose, 140 mCi/dose, 150 mCi/dose, or 160 mCi/dose. In still another embodiment, the activity range for a unit dose containing the radiopharmaceutical composition is from about 130 mCi/dose to about 150 mCi/dose. In yet another embodiment, the activity range for a unit dose containing the radiopharmaceutical composition is about 146 mCi/dose.

In one embodiment, the radiopharmaceutical composition is prepared in bulk, wherein the radioactivity of the bulk solution is from about 1 mCi to about 100,000 mCi, 1 mCi to about 90,000 mCi, 1 mCi to about 80,000 mCi, 1 mCi to about 70,000 mCi, 1 mCi to about 60,000 mCi, 1 mCi to about 50,000 mCi, 1 mCi to about 40,000 mCi, 1 mCi to about 10,000 mCi, from about 100 mCi to about 9,000 mCi, from about 1,000 mCi to about 8,000 mCi, from about 3,000 mCi to about 5,000 mCi, from about 1 mCi to about 5,000 mCi, from about 5,000 mCi to about 10,000 mCi, from about 1,000 mCi to about 30,000 mCi, from about 1,000 mCi to about 20,000 mCi, from about 10,000 mCi to about 40,000 mCi, from about 20 mCi to about 30,000 mCi, from about 5,000 mCi to about 30,000 mCi, or from about 10,000 mCi to about 15,000 mCi. In another embodiment, the radioactivity of the bulk solution is less than about 40,000 mCi, less than about 30,000 mCi, less than about 20,000 mCi, less than about 15,000 mCi, less than about 10,000 mCi, less than about 9,000 mCi, less than about 8,000 mCi, less than about 7,000 mCi, less than about 6,000 mCi, less than about 5,000 mCi, less than about 4,000 mCi, less than about 3,000 mCi, less than about 2,000 mCi, or less than about 1,000 mCi.

In yet another embodiment, the radiopharmaceutical composition is prepared with or without using a purification cartridge. In some embodiments, the radiopharmaceutical composition is obtained in high purity without purification.

In one embodiment, the radiopharmaceutical composition is chemically stable for up to about 40 hours, for up to about 41 hours, for up to about 42 hours, for up to about 43 hours, for up to about 44 hours, for up to about 45 hours, for up to about 46 hours, for up to about 47 hours, for up to about 48 hours, for up to about 49 hours, for up to about 50 hours, for up to about 51 hours, for up to about 52 hours, for up to about 53 hours, for up to about 54 hours, for up to about 55 hours, for up to about 56 hours, for up to about 57 hours, for up to about 58 hours, for up to about 59 hours, or for up to about 60 hours after formulation.

In one embodiment, the radiopharmaceutical composition is stored in a syringe. In still another embodiment, the radiopharmaceutical composition is stored in a glass vial.

### EXAMPLES

The following examples provide manufacturing, stability, and radiochemical purity data for various formulations.

### Example 1

The drug product is a sterile-filtered radiopharmaceutical solution containing Cu-64 PSMA I&T injection. The drug product is formulated in an ascorbic acid solution to target a maximum dose of 9 mCi (+/−10%) calibrated to a specific reference time.

Cu-64 PSMA I&T Injection is stored at controlled room temperature 200 to 25° C. (68° to 77° F.) in the original package shielding to protect persons from ionizing radiation. Temperature excursions between 15° C.-30° C. (59° F.-86° F.) are permitted during storage. The expiration is approximately 1900 CT day of calibration and the shelf life for the drug product is approximately 49-51 hours.

### Example 2—Examples of Cu-64 PSMA I&T Unit Doses

An example of a Cu-64 PSMA I&T Unit Dose formulated for a 9 mCi dose at calibration time is provided in Table 4.

TABLE 4
Cu-64 PSMA I&T 9 mCi Unit Dose

| | | | Quantity | | Quantity | |
|---|---|---|---|---|---|---|
| | | | per | | per unit | |
| Name of | | Quality | milliliter | | dose | |
| ingredient(s) | Function | standard | (1 mL) | | (4 mL) | |
| ⁶⁴Cu-PSMA I&T | Drug | GMP | 2.25 | mCi^{a} | 9 | mCi^{a} |
| | substance/active | | | | | |
| | pharmaceutical | | | | | |
| | ingredient | | | | | |
| Ascorbic Acid^{b} | Excipient | USP | 37 | mg | 148 | mg |

| | | | | |
|---|---|---|---|---|
| (Sodium | (pH buffer and | | | |
| Ascorbate) | stabilizer) | | | |
| Sterile Water for | Excipient | USP | q.s. | q.s. |
| Injection | (solvent) | | | |

q.s: quantum satis (as much as is enough)
^{a}At calibration;
^{b}Sodium ascorbate is used to formulate solutions for drug product manufacture. The concentration of sodium ascorbate is determined in the drug product and is listed in the specification as ascorbic acid content (Target: 37 mg/mL).

An example of a Cu-64 PSMA I&T Unit Dose formulated for a 5 to 9 mCi dose at calibration time is provided in Table 5.

TABLE 5
Cu-64 PSMA I&T 5-9 mCi Unit Dose

| | | | Quantity | | Quantity | |
|---|---|---|---|---|---|---|
| Name of | | Quality | per milliliter | | per unit dose | |
| ingredient(s) | Function | standard | (1 mL) | | (up to 4 mL) | |
| ⁶⁴Cu-PSMA | Drug | GMP | 1.25-2.25 | mCi^{a} | 5-9 | mCi^{a,b} |
| I&T | substance/ | | | | | |
| | active | | | | | |
| | pharmaceutical | | | | | |
| | ingredient | | | | | |
| Ascorbic | Excipient | USP | 37 | mg | 84-148 | mg^{b} |

| | | | | |
|---|---|---|---|---|
| Acid^{c} | (pH buffer and | | | |
| (Sodium | stabilizer) | | | |
| Ascorbate) | | | | |
| Sterile Water | Excipient | USP | q.s. | q.s. |
| for Injection | (solvent) | | | |

q.s: quantum satis (as much as is enough)
^{a}At calibration.
^{b}The patient dose will be either 5 mCi, 7 mCi or 9 mCi; Sodium ascorbate is used to formulate solutions for drug product manufacture. The concentration of sodium ascorbate is determined in the drug product and is listed in the specification as ascorbic acid content (Target: 37 mg/mL).

### Example 3—Manufacture of Copper Cu-64 PSMA I&T Injection Using a Purification Cartridge

Radiolabeling: A buffered solution containing approximately 1 mg/mL PSMA I&T, 54-56 mg/mL of sodium acetate trihydrate, and 3-5 mg/mL of gentisic acid was prepared (pH 4-5.5). To radiolabel, the PSMA I&T solution was mixed with a solution of [⁶⁴Cu]CuCl₂ to target the desired specific activity, and the mixture was held at ambient temperature or gently heated for 5-30 min. After the reaction was complete, the crude reaction mixture was loaded onto a Cis SPE cartridge, the cartridge was rinsed with water, and the [⁶⁴Cu]Cu-PSMA I&T drug substance was eluted in a solution of 50% ethanol.

Final drug product formulation: The drug substance solution was passed through a sterilizing filter and collected in the final multi-dose vial, wherein the radioactive concentration was assessed and could be diluted with sodium ascorbate buffer to prepare the final drug product at a concentration of 1.25-2.5 mCi/mL (at calibration). Prior to release, the drug product was tested to ensure it meets acceptable quality and safety requirements. The process described herein can be performed manually, or using an automated synthesis unit.

### Example 4—Removal of Purification Cartridge

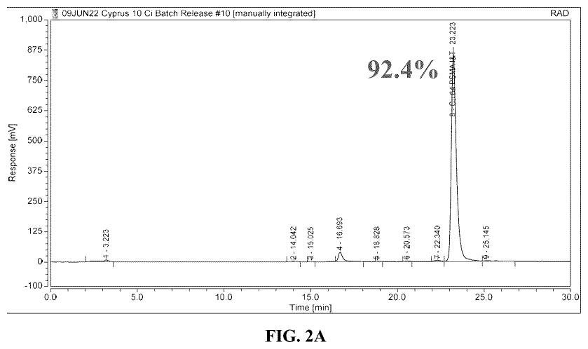

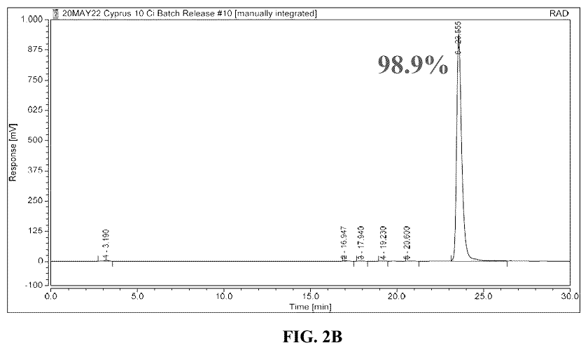

Two 10 Ci batches of copper Cu-64 PSMA I&T were prepared, wherein the primary difference between them was removal of the purification step. During purification, a solid phase extraction cartridge was used to purify the product by removing radiolabeling buffer and remaining ⁶⁴Cu copper chloride starting material; however, this step requires concentration of the batch activity in a small volume (i.e., <1 mL), which can encourage radiolytic damage and result in detrimental impacts to radiochemical purity (RCP). This phenomenon was observed experimentally when comparing the radiochemical purity of copper Cu-64 PSMA I&T prepared with and without purification (FIG. 2). The removal of the purification step yielded a product with a higher RCP of 98.9% (FIG. 2B) versus 92.4% (FIG. 2A). The difference between the two purity profiles was that the primary radiolytic impurity (t_{R}=16.8′-17.2′) was largely absent in the non-purified product. In both formulations, the RCP of individual dose vials prepared thereof was maintained over a period of 51 h post-formulation (i.e., 51 h after end of synthesis).

All references cited herein are hereby incorporated by reference. The foregoing is offered primarily for purposes of illustration. It will be readily apparent to those skilled in the art that further drugs can be included, and that the components, additives, proportions, methods of formulation, methods of use, and other parameters described herein can be modified further or substituted in various ways without departing from the spirit and scope of the invention.

What is claimed is:

1. A radiopharmaceutical composition comprising a Copper Cu-64 PSMA I&T solution for injection comprising:
(a) Copper Cu-64 PSMA I&T in an amount from about 1 mCi to about 15 mCi per unit at the time of dose,
(b) gentisic acid in a concentration from about 0 mg/mL to about 3.5 mg/mL, and
(c) sodium acetate in a concentration of about 1 mg/mL to about 5 mg/mL, and wherein the radioactivity concentration of the radiopharmaceutical composition is from about 1 mCi to about 36 mCi.

2. The radiopharmaceutical composition of claim 1, wherein the composition has a radioactivity of less than about 10 mCi.

3. The radiopharmaceutical composition of claim 1, wherein the composition comprises Copper Cu-64 PSMA I&T in an amount of about 1 mCi, 2 mCi, 3 mCi, 4 mCi, 5 mCi, 6 mCi, 7 mCi, 8 mCi, 9 mCi, or 10 mCi per unit at the time of dose.

4. The radiopharmaceutical composition of claim 1, wherein the composition comprises gentisic acid in an concentration of about 1.3 mg/mL to about 3.3 mg/mL.

5. The radiopharmaceutical composition of claim 1, wherein the composition comprises sodium acetate in an concentration of about 2 mg/mL to about 4 mg/mL.

6. The radiopharmaceutical composition of claim 1, wherein the composition comprises sodium acetate in an concentration of about 2.5 mg/mL to about 3.5 mg/mL.

7. The radiopharmaceutical composition of claim 1, wherein the composition is sterile.

8. The radiopharmaceutical composition of claim 1, wherein a unit dose of the composition has a volume from about 1 mL to about 10 mL.

9. The radiopharmaceutical composition of claim 1, wherein a unit dose of the composition has a volume of about 4 mL.

10. The radiopharmaceutical composition of claim 1, wherein the composition has a radiochemical purity of at least 99%, as measured by HPLC at 0 hour post EOS.

11. A radiopharmaceutical composition for use in the diagnosis of prostate cancer in a human subject comprising, at the time of dose:
(a) Copper Cu-64 PSMA I&T in an amount from about 5 mCi to about 9 mCi,
(b) ascorbic acid in an amount of about 84 mg to about 148 mg, and
(c) water in a quantum satis for injection into the human patient.

12. The radiopharmaceutical composition of claim 11, wherein the composition has a radioactivity of less than about 10 mCi.

13. The radiopharmaceutical composition of claim 11, wherein the pH of the composition is from about 5.5 to about 7.5.

14. The radiopharmaceutical composition of claim 11, wherein the composition is sterile.

15. The radiopharmaceutical composition of claim 11, wherein the composition has a volume from about 1 mL to about 10 mL.

16. The radiopharmaceutical composition of claim 11, wherein the composition has a volume of about 4 mL.

17. The radiopharmaceutical composition of claim 11, wherein the composition has a radiochemical purity of at least 95%, as measured by HPLC.

18. The radiopharmaceutical composition of claim 11, wherein the composition has a radiochemical purity of at least 96%, as measured by HPLC.

19. The radiopharmaceutical composition of claim 11, wherein the composition has a radiochemical purity of at least 97%, as measured by HPLC.

20. The radiopharmaceutical composition of claim 11, wherein the composition has a radiochemical purity of at least 98%, as measured by HPLC.

21. The radiopharmaceutical composition of claim 11, wherein the composition has a radiochemical purity of at least 99%, as measured by HPLC.

22. A radiopharmaceutical solution for use in the diagnosis of prostate cancer in a human subject comprising:
(a) Copper Cu-64 PSMA I&T in an amount from about 1 mCi to about 15 mCi per unit at the time of dose,
(b) gentisic acid in a concentration from about 0 mg/mL to about 3.5 mg/mL, and
(c) sodium acetate in a concentration of about 1 mg/mL to about 5 mg/mL.

23. The radioactive solution of claim 22, wherein the radioactivity concentration of the radiopharmaceutical solution is up to 36 mCi.

24. The radiopharmaceutical solution of claim 22, wherein the solution comprises Copper Cu-64 PSMA I&T in an amount of about 1 mCi, 2 mCi, 3 mCi, 4 mCi, 5 mCi, 6 mCi, 7 mCi, 8 mCi, 9 mCi, or 10 mCi per unit at the time of dose.

25. The radiopharmaceutical solution of claim 22, wherein the solution comprises gentisic acid in an concentration of about 1.3 mg/mL to about 3.3 mg/mL.

26. The radiopharmaceutical solution of claim 22, wherein the solution comprises sodium acetate in an concentration of about 2 mg/mL to about 4 mg/mL.

27. The radiopharmaceutical solution of claim 22, wherein the solution comprises sodium acetate in an concentration of about 2.5 mg/mL to about 3.5 mg/mL.

28. The radiopharmaceutical solution of claim 22, wherein the pH of the solution is from about 5.5 to about 7.5.

29. The radiopharmaceutical solution of claim 22, wherein the solution has a radiochemical purity of at least 99%, as measured by HPLC at 0 hour post EOS.

30. The radiopharmaceutical solution of claim 22, wherein the solution has a radiochemical purity of at least 99%, as measured by HPLC at the time of dose.
